(19) United States
(12) Patent Application Publication
(10) Pub. No.: US 2016/0022145 A1
(43) Pub. Date: Jan. 28, 2016

(54) APPARATUS AND METHODS FOR REMOTE MONITORING OF PHYSIOLOGICAL PARAMETERS

(71) Applicant: Kirill MOSTOV, Berkeley, CA (US)

(72) Inventor: Kirill Mostov, Berkeley, CA (US)

(21) Appl. No.: 14/775,617

(22) PCT Filed: Mar. 12, 2014
(86) PCT No.: PCT/US14/25063

§ 371 Date: Sep. 11, 2015

Related U.S. Application Data
(60) Provisional application No. 61/786,535, filed on Mar. 15, 2013.

(51) Int. Cl. A61B 5/00; A61B 5/1455

(52) U.S. Cl. CPC A61B 5/0026; A61B 5/14552; A61B 5/7228; A61B 5/0004; A61B 2562/0219

(57) ABSTRACT

The invention relates to an apparatus for remote monitoring physiological parameters, comprising: a radar transmitter for transmitting radio frequency signal towards a human body; and a radar receiver for receiving frequency signals reflected from a human body. An accelerometer is adapted to be placed on a human body, and a signal processor, which is configured for extracting and processing physiological parameters of the at least one human body from the inputted signals of the receiver and accelerometer. A specific embodiment of the apparatus relates to a radar and a wireless communication channel controller for receiving data from patients. Patients have accelerometers attached to their bodies and the patients can be without motion or move freely within a room. For each patient, the device determines breathing and pulse rate; cardiac performance and the patient identification by automatically setting up a match between the determined parameters and each patient's ID.

### RELATED APPLICATIONS

This application claims priority from U.S. Provisional Patent Application No. 61/786.535. entitled “A Device For Remote Contactless Definition of State of The Person”, filed 15 Mar. 2013, which is incorporated herein by reference.

### FIELD OF THE INVENTION

This invention relates to devices and methods for remote contactless monitoring physiological parameters of individuals.

### BACKGROUND OF THE INVENTION

One of the problems within the framework of patient's treatment is monitoring of his/her vital parameters. The primary requirements to the monitoring are: it needs to be implemented permanently, in a most comfortable way for a patient, and the collected data shall be accurate.

Previous attempts to obtain vital biological parameters of a patient were done with a help of expensive and clumsy devices. Multiple sensors have been placed on a patient body; often these caused wire mishmash.

One prior medical device is designed to be operated by a patient, without the need for extensive training. It monitors the patient's heart rhythm and determines if a patient is likely experiencing supraventricular arrhythmia. This device may be used periodically, for regular checks of a patient's heart rhythm, or it may be used in a continuous, uninterrupted manner, for constant monitoring a patient's heart rhythms. When used by a patient under the guidance and supervision of medical professionals, the device can aid in the detection of intermittent supraventricular arrhythmia, can assist in determining the duration of the arrhythmia, and can assist in customizing the appropriate dosage of medication to fit the patient's specific needs. However, this device uses sensors placed on a human body and connected to its control module by the cable. This is inconvenient for the patient, and unreliable.

Another prior medical device (U.S. Pat. No. 7,507,203; Sebastian at al.) uses laser radar for pure remote operation. The signal is radiated by a radar transmitter, and the reflected signal is captured by a receiver. A signal processor calculates the range to a patient and the range rate of the patient, using a reference signal from the transmitter. In one embodiment a frequency modulated optical signal is used, that simplifies further calculation through the determination of the modulation frequency difference between the reference signal and reflected signal. At the processing stage, the range between the device and the target is defined, as well as the range rate. Further processing of the composite signal with the exclusion of the range component allows to determine periodic components that characterize physiological parameters of a subject, including cardiovascular functions like heart rate, heart rate variability, pulse transit time, respiratory functions like respiration rate, respiratory effort, physical activities, etc.

The medical device discussed above, although having the ability to provide some physiological parameters, has serious drawbacks. These include:
inability to monitor several subjects,
a special requirement to provide reflecting elements on a subject clothes, otherwise the optical reflection is inefficient,
high cost of optical elements included in the device,
complete inability to operate through a barrier,

The objective of the present invention is to provide an apparatus for remote monitoring physiological parameters and psychological state of one or several individuals, even through a barrier, capable of monitoring several subjects and free from expensive components such as optics. Another objective is to provide a compact apparatus, which can be hand-held, not requiring complicated connection and installation. A further objective is to provide an apparatus, which can remotely monitor heartbeat, respiration rate, blood pressure, vasomotor fluctuations data, muscle tone, blood flow to the organs and Oxygen saturation.

### SUMMARY

The invention relates to an apparatus for remote monitoring physiological parameters, comprising a transmitting antenna for radiation of a radio frequency signal towards at least one human body, and at least one radar receiver for receiving a signal reflected from the at least one human body; and a signal processor. The radar receiver comprises a receiving antenna positioned at a predefined distance from the transmitting antenna, the apparatus further comprising at least one accelerometer adapted to be placed on a human body, and a signal processor. The apparatus further comprises at least one accelerometer adapted to be placed on a human body, and a signal processor, wherein respective outputs of the radar receiver and accelerometer are connected to the input of the signal processor which is configured for extracting and processing physiological parameters of the at least one human body from the inputted signals of the receiver and accelerometer. This embodiment provides monitoring physiological and psychological state of one or more individuals who may stay motionless or move within a room,

The accelerometer can contain a wireless transmitter, the signal processor can contain a wireless receiver, and the output of the accelerometer can be connected to the input of the signal processor through a wireless communication channel be connected to the signal processor through a wireless channel. This provides the convenience for the patient and excludes damaging the communication wires.

Preferably, the apparatus comprises at least two radar receivers, wherein their respective receiving antennae are positioned at a predetermined distance from one another and from the transmitting antenna.

The radar receiver can comprise a clocked amplifier having its input connected to the receiving antenna.

Preferably, the transmitter is adapted to produce frequency modulated signals in the form of a train of pulses with a predefined delay between pulses. The pulses can have duration of half a period of modulation frequency variation.

The present apparatus can comprise a digital-to-analog converter (DAC), whose input is connected to the output of the signal processor, and the output connected to the frequency deviation control of the transmitter. This embodiment, apart from the minimization of the subject radiation, provides the highest signal-to-noise ratio,and thus the highest possible precision.

Further, the invention relates to a method for determination of the distance from each receiving antenna to a body using the above apparatus, wherein the emitted frequency and the received frequency are measured at the same moment, the difference between these frequencies is multiplied to the modulation frequency sweep period, and divided by the modulation frequency swing, and the result is scaled by the multiplication to one fourth of the speed of light in the air. The method is based on the comparison of the modulation frequency of the reference signal and the reflected signal, where the modulation frequency has a linear dependence on time. This method is very simple and provides high resolution of the distance, that is about 1 cm.

Further, the invention relates to a method for determination of the azimuth to each body of the apparatus of claim 1, wherein a phase shift is measured between the harmonic components selected for the same human body, received from two receiver channels; and the required azimuth is obtained as the arc sine of the said phase shift multiplied to the radar wavelength and divided by the distance between the receiving antennae. The method is based on the measurement of the phase shift between harmonic components received from two radar receivers. This method is simple, and provides the resolution better than 1 degree of arc.

### BRIEF DESCRIPTION OF DRAWINGS

FIG. 1 is the general block diagram of the apparatus for determination of personal state, according to one embodiment.

FIG. 2 is the diagram of the apparatus main operation stages for determination of personal state, according to one embodiment.

FIG. 3 explains the signal processing procedure for determination of range to the patient, according to one embodiment.

FIG. 4 illustrates the processing result for a signal reflected from multiple objects, according to one embodiment.

FIG. 5 explains the signal processing procedure for determination of the patient azimuth, according to one embodiment.

FIG. 6 explains the use of triangulation method for determination of the patient azimuth, according to one embodiment.

FIG. 7 illustrates the typical signal when determining heart and respiration rates, according to one embodiment.

FIG. 8 illustrates data processing approach for psycho-physiological parameters determination, according to one embodiment.

FIG. 9 illustrates an exemplary waveform describing heart-muscle operation, according to one embodiment.

FIG. 10 illustrates an exemplary finger clip, according to one embodiment.

FIG. 11 illustrates the general block diagram of the sleep apnea monitor with a controlled key and explains how gating is used to decrease the power radiated by the device.

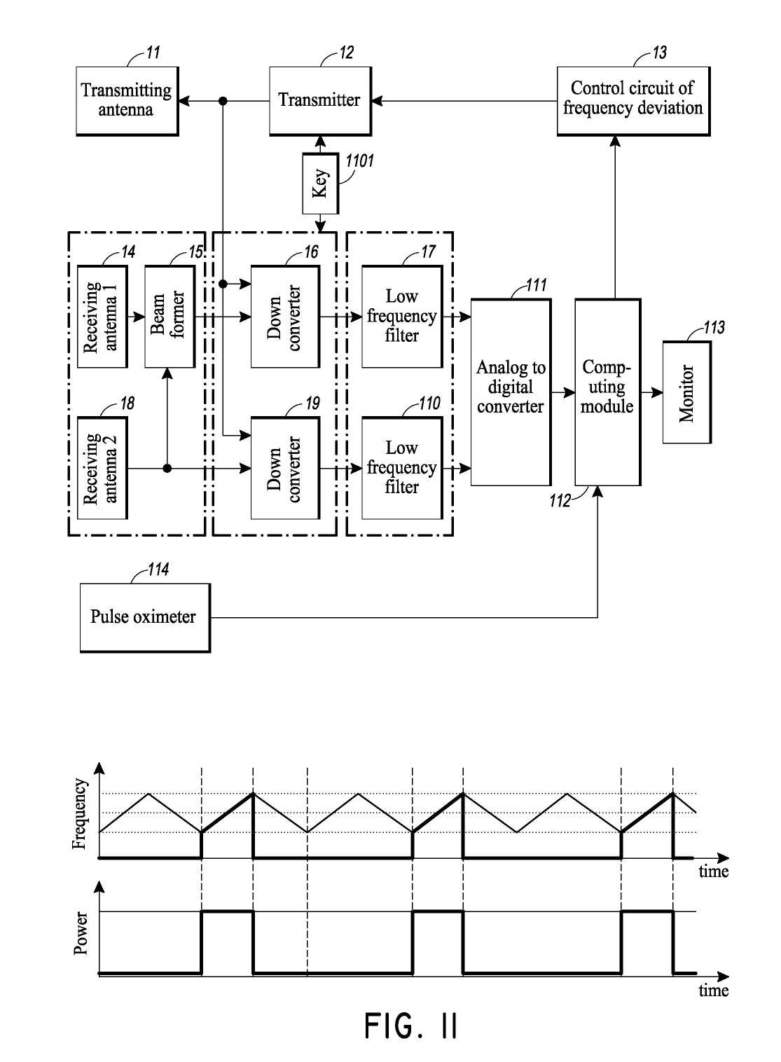

FIG. 12 illustrates the general block diagram of the sleep apnea monitor with a digital to analog converter installed between the computing module and the transmitter and explains how the converter is used to decrease the device's radiated power.

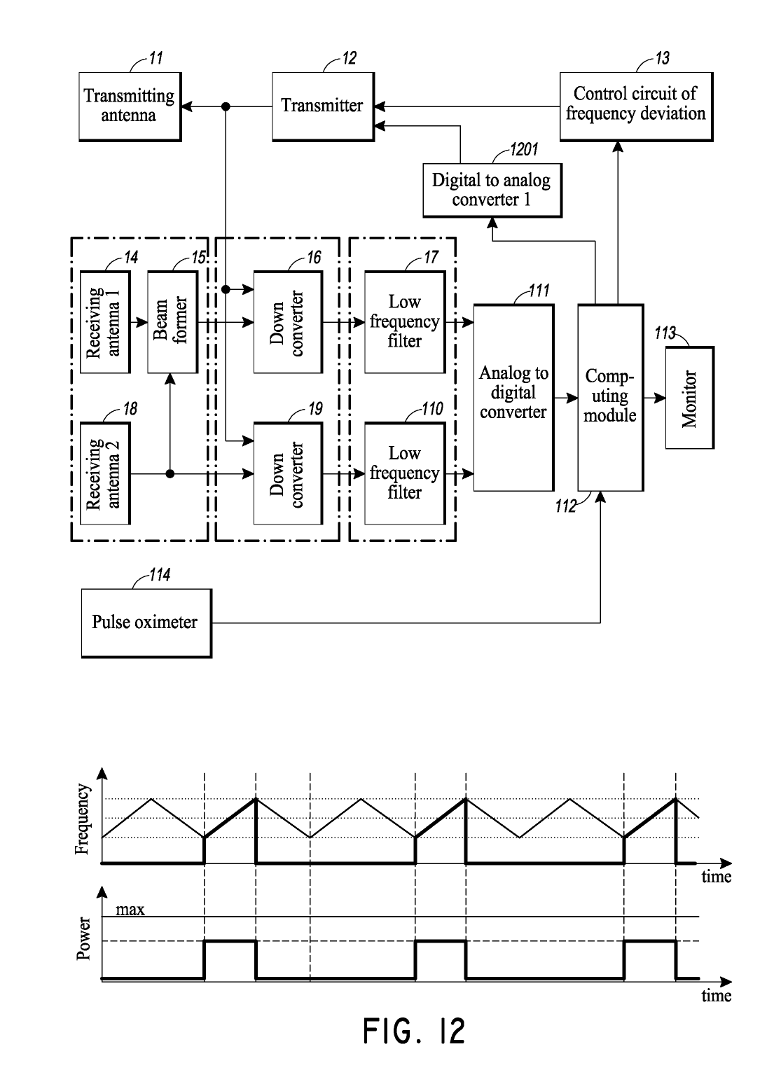

FIG. 13 illustrates the general block diagram of the sleep apnea monitor with a digital to analog converter installed between the computing module and the control circuit of frequency deviation and explains the method how controlling the radiated signal modulation frequency is used to decrease the radiated signal power.

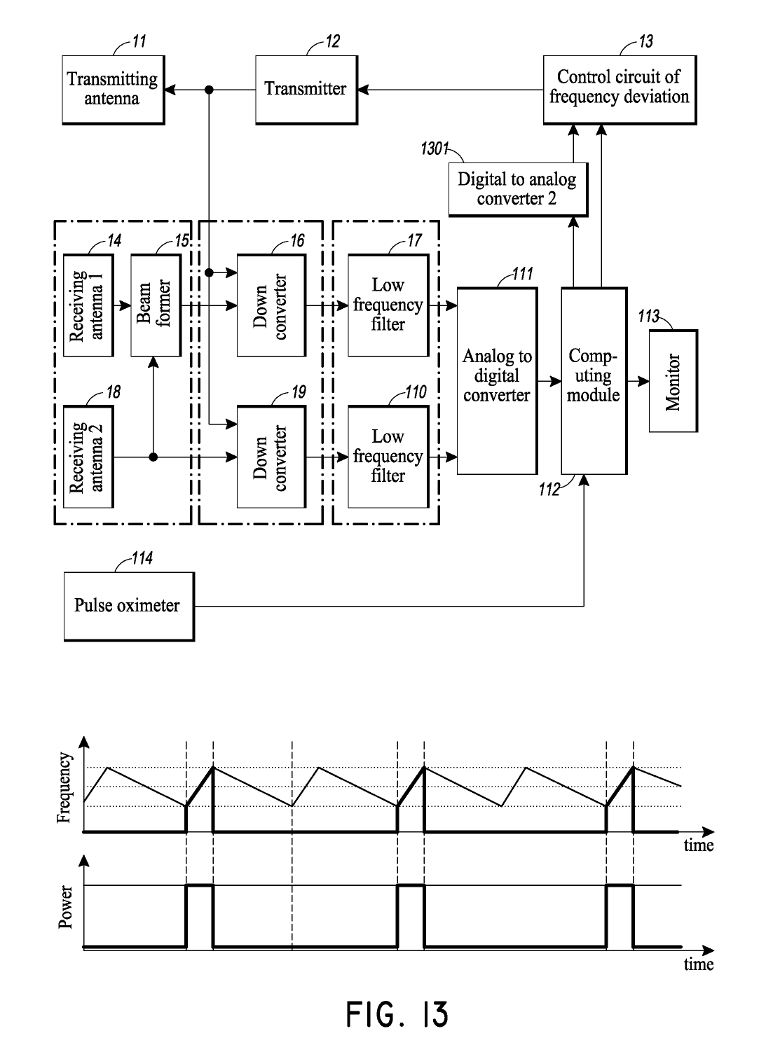

FIG. 14 illustrates the general block diagram of the sleep apnea monitor with an aggregate of components for decreasing the radiated signal power.

FIG. 15 illustrates the general block diagram of a modified apparatus for determination of personal state, wherein the radar operates in a self-adaptable mode.

FIG. 16 illustrates the general block diagram of the vital signs (VS) module.

FIG. 17 illustrates the general block diagram of the Data Processing Unit,

FIG. 18 illustrates the process in the Motion Trajectory Determination Unit.

FIG. 19 illustrates the process in the Motion Compensation Unit.

FIG. 20 schematically illustrates the structure of the apnea determination unit.

FIG. 21 illustrates the process in the Sleep/Wake Deter ruination Unit,

FIG. 22 illustrates the Determination of Sleep and Wake Modes.

FIG. 23 illustrates the process in the Plethysmogram-Based Parameter Formation Unit.

FIG. 24 illustrates the general block diagram of the apparatus for remote non-contact monitoring of physiological and psychological state of individuals, based on the UWB-radar technology.

FIG. 25 illustrates a Doppler radar system.

### DETAILED DESCRIPTION

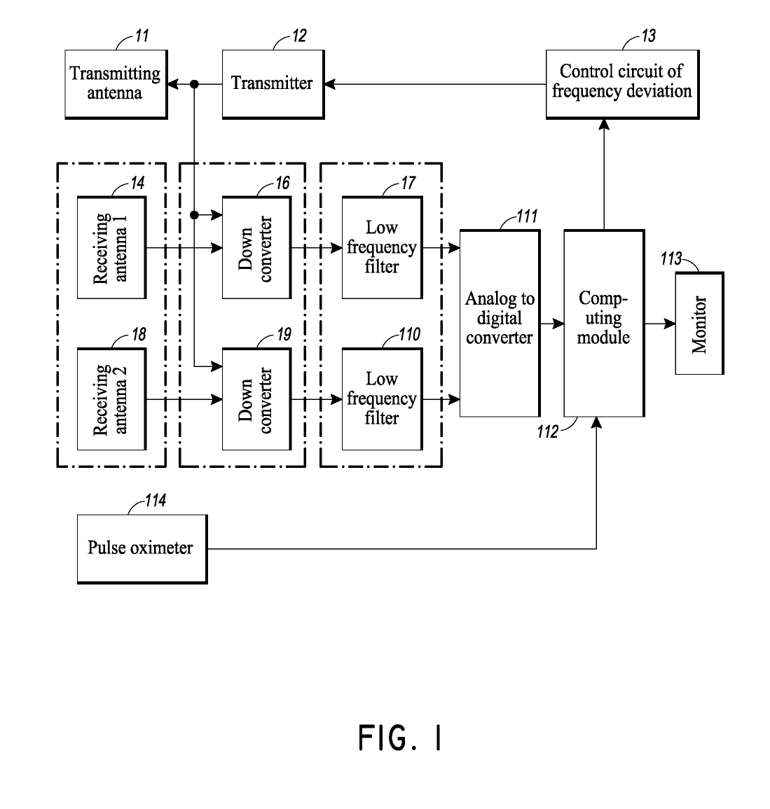

In FIG. 1, a general block diagram of the apparatus is presented. The apparatus generally consists of a transmitter (12), a transmitting antenna (11), a receiver comprising two receiving antennae (14, 18), two down converters (16, 19), two low frequency filters (17, 110), analog to digital converter (111), a processor or computing module (112), control circuit of frequency deviation (13) and monitor (113).

The transmitter (12) generates the signal, which is radiated by the transmitting antenna (11), and simultaneously delivers the signal to the down converters (16, 19); this signal is further used as a reference signal. The Signal reflected from the exposed object is received by the receiving antenna_1 (14) and receiving antenna_2 (18) and is delivered to the down converters (16, 19) via individual channels. Each down converter multiplies the reference and received signals. After conversion, the signals are processed in the low frequency filters (17, 110), whereby the low-frequency component is isolated in each channel and then delivered to the analog-to-digital converter (111). The digital data is then delivered to the computing module (112), which makes calculations and then determines the psychophysiological and mental state of the exposed subject and displays the information about the subject's state on the monitor screen (113).

The apparatus is capable of determining the psychophysiological and mental state of a single individual with just one receiving antenna.

When there are more than one individual within the coverage area of the device, identification is required, which signal belongs to which target. When just one receiving antenna is used, the device is capable of determining the range to target. The device is capable of determining the azimuth of a target; for this purpose, it contains two reception antennae.

The range and azimuth data make it possible to determine, which signal corresponds to which subject, and separate the signals to determine the state of multiple persons simultaneously.

In addition, the range and azimuth data allow separation of signals reflected by various body organs of a given subject, making possible to acquire information on the peripheral hemodynamics in various organs (heart, stomach, liver, etc.) individually. Such information is very important for medical surveillance over the patient.

The apparatus is capable of receiving signals from pulse oximeter (114) placed on a patient finger. The pulse oximeter signal is transmitted to the main unit via cable or for convenience via wireless channel.

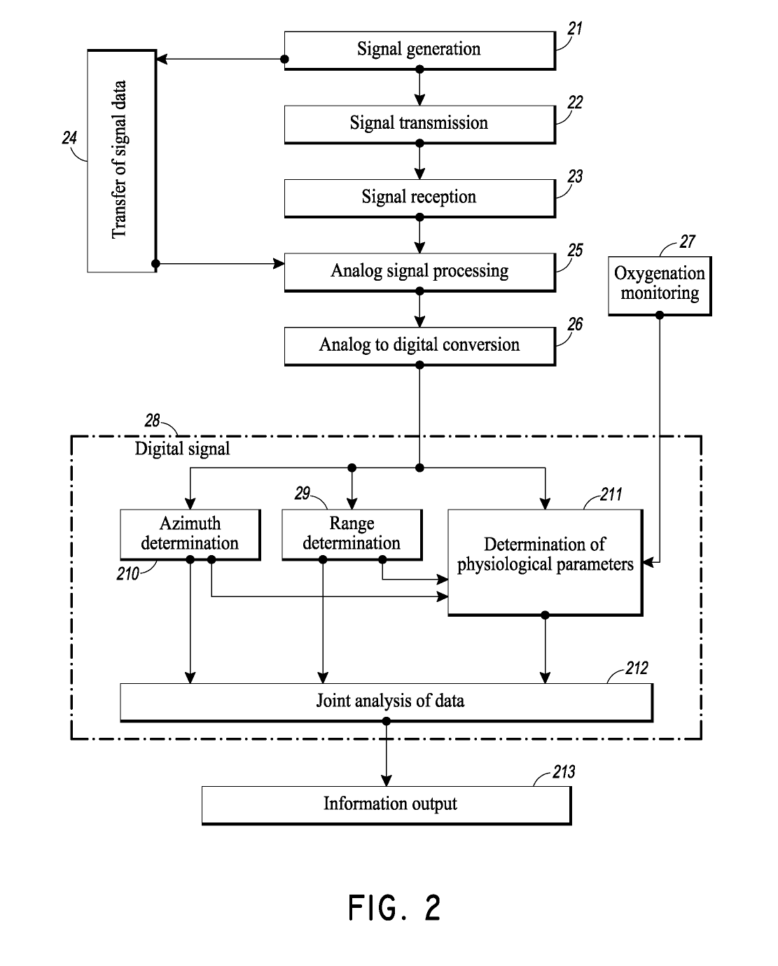

The diagram of the apparatus main operation stages for determination personal state is given in FIG. 2. When the device is on, the signal is generated 21, transmitted 22 and received 23. Analog processing 25 of signals received via individual channels from each of the receiving antennae uses the reference signal 24 that is supplied from the transmitter 12 on FIG. 1 to the down converters 16 and 19.

After the analog signal processing in the low frequency filters block the signals are digitized 26, and the digital signal processing 28 takes place. The process of digital signal processing includes calculations that make it possible to
determine the range to target 29, target position azimuth 210,
determine physiological and psychological parameters of subjects 211.

For determination of the parameters 211, data from a subject's oxygenation monitor 27 shall be used. When the state monitoring is carried out for multiple subjects, the data on range 29 and azimuth 210 is used for signal separation in order to determine the physiological and psychological parameters 211 for individual subjects. Calculation results are jointly analyzed 212 and the information is displayed 213 on the screen.

For an object position measurement in two-dimensional coordinates, the range to an object and an object azimuth are calculated.

To provide range calculation, the high-frequency return signal received by each reception antenna is compared against the reference signal.

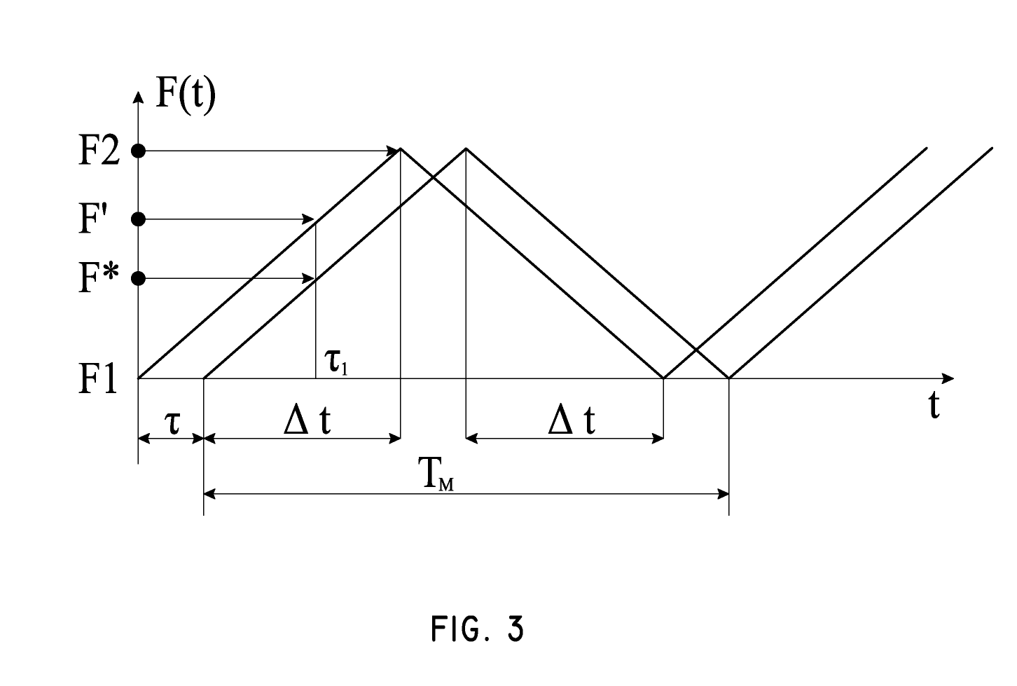

FIG. 3 explains the range definition algorithm that uses the frequency of the received signal. The transmitting antenna emits the signal F′. The down converter converts the received analog signal. The low-frequency filter makes it possible to isolate the low-frequency component of the analog signal carrying the target range data. The analog-to-digital conversion allows input signal to be sampled to produce a set of N points for the signal received by each antenna. At that:

$$N = ( \frac{{T}_{M}}{2} - τ ) \cdot {F}_{d} ,$$

wherein:
T_{M}/2=frequency sweep period: frequency change from F1 to F2;
F=analog input sampling rate;
τ=time interval between signal transmission and signal reception.

The emitted signal F upon reaching the target at the distance R from the device, is reflected back and subsequently received by both reception antennae. Thus, the emitted signal will acquire a time delay: τ=2*R/c, where c—speed of light in the air. The receiver determines frequency F_{R}, equal to the difference of frequencies (F) (emitted) and (F*) (received) during the moment of tim T₁. Thus:

$${F}^{′} = {F}_{1} + \frac{2 ( {F}_{2} - {F}_{1} )}{{T}_{M}} {τ}_{1} ;$$

$${F}^{*} = {F}_{1} + \frac{2 ( {F}_{2} - {F}_{1} )}{{T}_{M}} ( {τ}_{1} - τ )$$

$${F}_{R} = {F}^{′} - {F}^{*} = \frac{4 ( {F}_{2} - {F}_{1} ) R}{c {T}_{M}}$$

$$R = \frac{c {T}_{M} ( {F}^{′} - {F}^{*} )}{4 ( {F}_{2} - {F}_{1} )}$$

R is he required distance to the target.

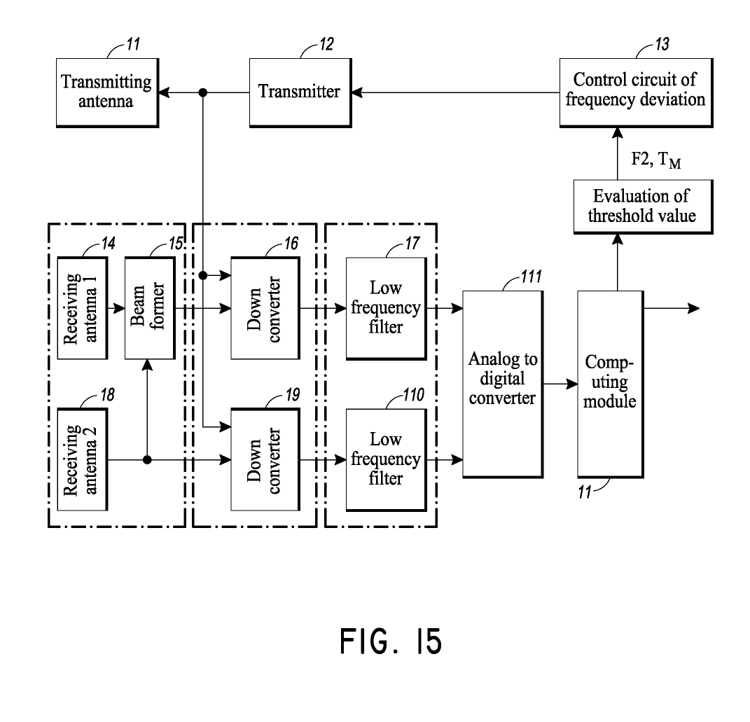

A modified device for determination of personal state, wherein the radar operates in a self-adaptable mode is presented in FIG. 15. In this apparatus a module for the evaluation of threshold value is introduced between the control circuit 13 of frequency deviation and computing module 11. This modification allows optimising the frequency range of the radar in relation to the distance to object, and thus reduce the noise level, i.e. improve the signal-to-noise ratio, and finally improve the range determination precision.

In operation, the radar is initially in its regular mode. Computing module 13 evaluates amplitudes for each range sample. The evaluations thus obtained are input to the module for evaluation of the threshold value. The module for evaluation of the threshold value, by comparing the amplitudes for each range sample, determines the sample having the biggest number (Rmax), wherein the amplitude for this sample exceeds a predetermined threshold. It shall, however, be clear that the maximum rang: to the object currently does not exceed the Rmax value. Thus, it is expedient to adjust the radar parameters F2 and TM so as to the maximum range of the radar does not exceed the Rmax value, and the frequency band is reduced proportionally. The module calculates the optimal values F2 and TM, optimal in view of the Rmax value, and delivers these values to the frequency deviation control circuit 13. In the control circuit 13 a frequency sweep for the signal emitted by the radar is formed, using the the values F2 and TM.

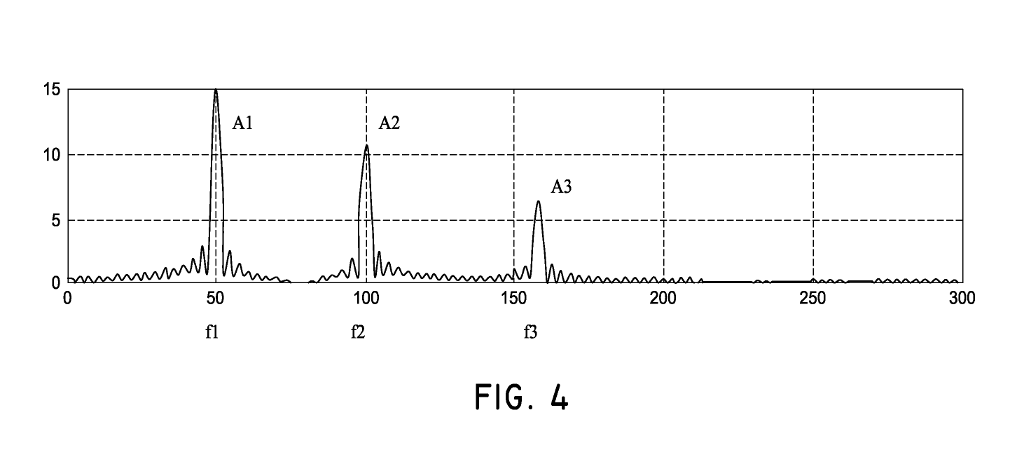

FIG. 4 illustrates the processing result for a signal reflected from multiple objects. When there are multiple targets within the coverage area of the device, the low-frequency signal comprises the appropriate number of harmonic components, each with a frequency corresponding to the distance to a particular target or its part.

The azimuth determination is based on the orientation diagram. For each frequency shown in FIG. 4 and corresponding to the distance to a particular target, the signal amplitude Aj acquired by the receiving antenna_1 is compared with the signal amplitude Ak: acquired by the receiving antenna_2.

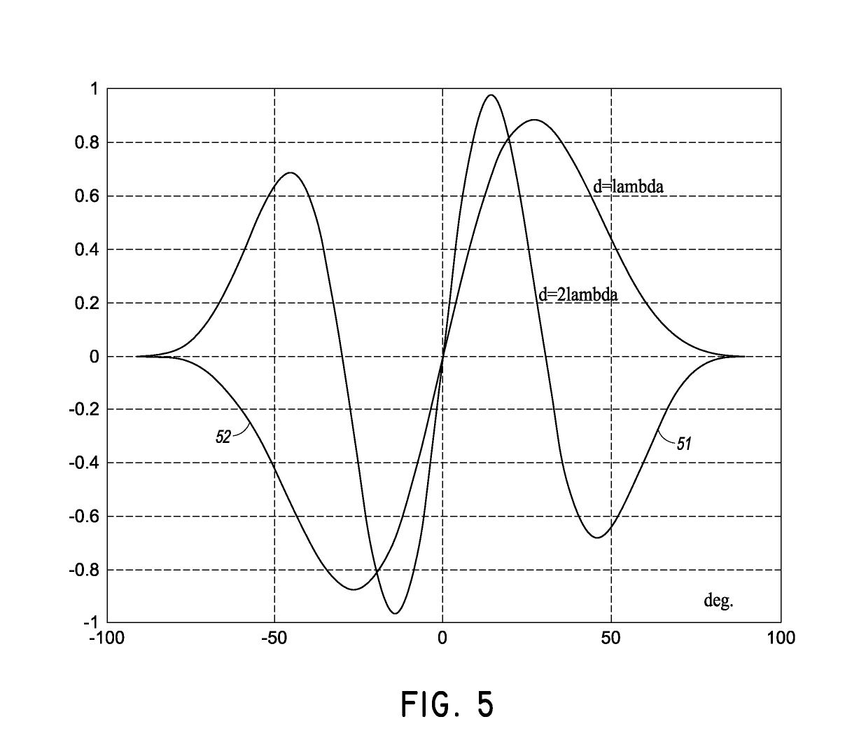

FIG. 5 demonstrates the orientation diagram of the azimuth determination. In the diagram legends, d is the distance between receiving antenna_1 and receiving antenna_2, and lambda is wavelength. Angles between the normal and the receiving antenna _2 in degrees are plotted on the X axis.

Within ±30° sector value of the signal is proportional to the value of azimuth (Plot 51). While the distance between antennae becomes smaller, the spectrum width increases, but the slope of curve decreases (Plot 52).

The azimuth determination is based on turnstile characteristic P=Aⱼ/Aₖ, P defines azimuth value. P doesn't depend on the distance to the target or on the effective radar cross section.

Within the linear part of the orientation diagram (FIG. 5) azimuth 0 is determined as θ=S·P, where S the slope of the curve of the turnstile characteristic. The described method is simple for calculation, but has a serious disadvantage azimuth to the target can be detected only when distance to the target is significantly bigger than the distance between the antennae.

There is another, more precise, method of azimuth determination. The high accuracy of distance measurement (r.m.s. error is about 1 cm) allows determining azimuth with the desirable accuracy while the distance between antennae is about 20 cm. Actually the distance between antennae is limited only by the device dimensions.

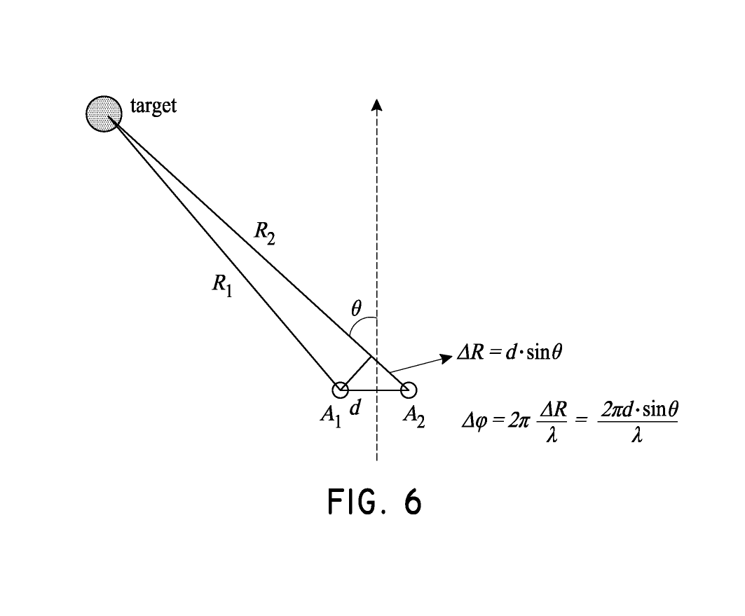

FIG. 6 explains the azimuth θ calculation procedure when the triangulation method is used.

Because of the difference of distances between the target and antenna_1 and antenna_2 (AR), the phase of the signal received by the reception antenna_1, will be delayed from the phase of the signal received by the reception antenna_2 by a value:

$$Δϕ = \frac{Δ R}{λ} 2 π = \frac{L \cdot sin θ}{λ} 2 π = k \cdot L \cdot sin θ$$

where

$$k = \frac{2 π}{λ} ,$$

λ—is the wavelength of the radiated (received) signal.

The value. θ can be obtained from these equations.

Processing periodic variations of the reflected signal a low measure and calculate remotely (even through opaque barriers) different rhythms of human body function. R is possible to measure these parameters by illuminating the entire group of people and selecting each person by its range and its azimuth. For determining rhythmic processes, the transmitter emits its signal during several seconds. Series of reflected signal magnitudes are exposed to FFT, which results signal spectrum for each target.

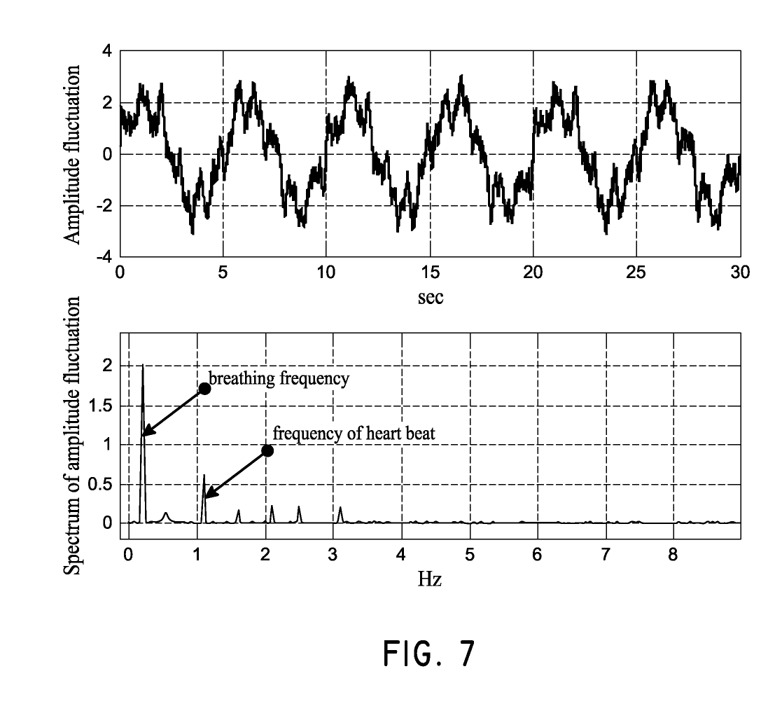

FIG. 7 demonstrates a typical waveform of amplitude fluctuations of a person and its spectrum. Psycho- and physiological parameters are determined through the analysis of this spectrum.

Different processes that take place inside a subject during monitoring have different impact on the parameters of the signal reflected from the subject. The frequency, amplitude and average value of the received signal are all changed. When analyzing the received signal, the characteristic waveform variations make it possible to identify symptoms of the processes.

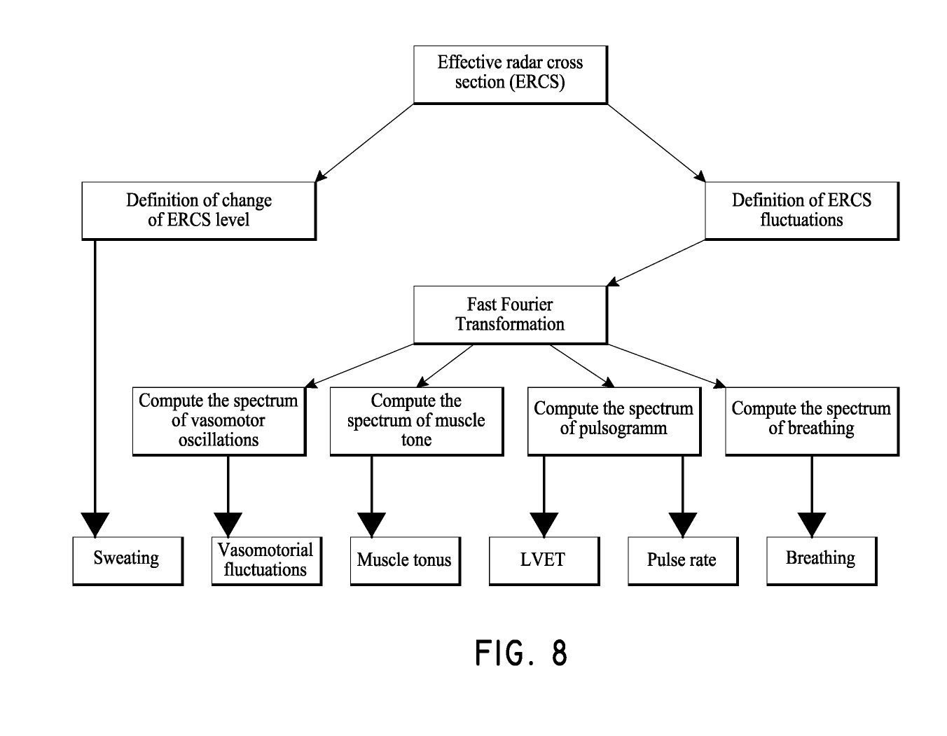

FIG. 8 illustrates a flow diagram of an exemplary process to determine psycho-physiological parameters, according to one embodiment. An analysis of the changes in the effective radar cross section (ERCS) determines the changes in perspiration. Fluctuations in ERGS provide information about plethysmogram, breathing, vasomotorial functions and muscle tonus. Each physiological parameter has its own fluctuation frequency. Typical vasomotorial signals are placed within the range 0.0017 . . . 0.017 Hz, the muscle tonus signals between are placed within the range 0.017 . . . 0.17 Hz, breathing signals are placed within the range 0.08 . . . 0.5 Hz, and heart beat signals within the range 0.67 . . . 4 Hz.

The received signal is affected by internal and external (with respect to the apparatus) electromagnetic fluctuations, which interfere with the useful signal and result in additional signal fluctuations known as “noise”. Individual processes related to the subject affect the signal in the same way as interference. For instance, the limb movement appear as high frequency/high amplitude “noise” in the radar signal. Sudden noise reduction is a sign of termination or absence of limb movements, which can be important in subject state monitoring.

The apparatus can be used for diagnostics of vascular diseases. For example, diabetes can lead to affection of lower limb vessels and development of diabetic foot. Timely examination of vessels allows anticipatory identification of factors predisposing the development of circulatory disturbance. Subject's leg vessels are periodically examined and the findings are recorded in a computer. The return signal parameters depend on the blood flow parameters and on the changes, if any, in limb tissue. In the next diagnostics the newly acquired return signal parameters are compared against the previous ones, which are stored in a data bank.

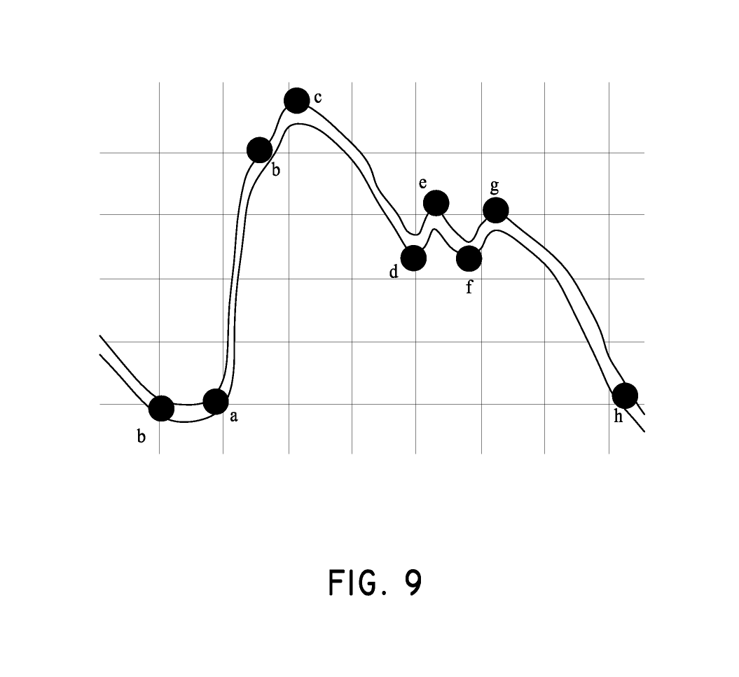

FIG. 9 illustrates an exemplary waveform describing heart-muscle operation, according to one embodiment. From the waveform constructed by the apparatus, left ventricular ejection time (LVET) and heart beat can be determined. LVET is the heart functional parameter (the rate of contraction of the left ventricle), which is known to be correlated to hostile intent.” The left ventricle is, in essence, the “pump”, which pushes blood on the “large circuit”. The right ventricle pumps blood on the smaller respiratory/lung circuit,

The apparatus detects plethysmogram in real time. A plethysmogram is a derived “measurement” of the heart activity. A plethysmogram can be used to evaluate the heart activity and compute LVET based on an analysis of the fluctuations in the amplitude of the reflected signal and the relative position of the characteristic points on the plethysmogram.

FIG. 9 shows a typical heart cycle (plethysmogram) having the following phases of interest: a-b-c is a systole phase with the increased pressure during heart muscle contraction; c-d is the phase of reduction of pressure at the tail end of systole; e is the phase of closing half moon valves; and f-t -h is the phase of reduction of blood pressure during diastole.

Phase a-b-c, the isometric contraction of the ventricle's systole, occurs with closed heart valves. The beginning of this phase coincides with the phase of abrupt increase in the internal to ventricles pressure. The derivative at the point ‘a’ can be used for the analysis of intensity and speed of ventricle operation. The amplitude of a-b-c correlates to the arterial pressure; one of the main parameters of heart operation. Measured peripheral blood pressure can be analyzed as low-pass-filtered arterial pressure.

Therefore, the operation of left ventricle can be characterized by (a) the heartbeat frequency; (b) the speed, with which the left ventricle muscle tissue is changing its tone, e.g. transitions from the relaxed to the contracted state; and (c) the blood pressure created by the left ventricle for opening the valve (instantaneous power of the pump with respect to one blood ejection from the ventricle).

Since the right ventricle operates at an order of magnitude lower power, the plethysmogram of the peripheral pulse provides rich source of information on the physiology of the left ventricle.

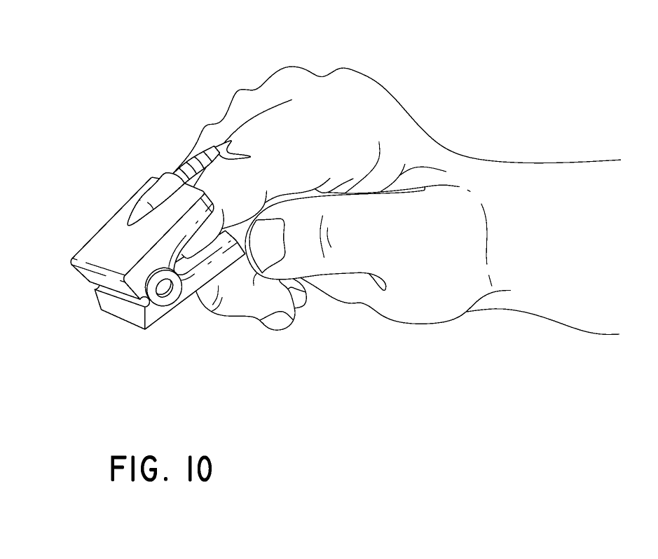

FIG. 10 illustrates an exemplary finger clip that is a pulse oximeter, with which the oxygenation in the patient is monitored. The acquired data is used for adjusting the subject's plethysmogram and physiological parameters.

The processing of physiological parameters makes it possible to determine hostile intent of the analyzed target.

The psychological condition of a human being can be characterized by the values of physiological parameters as illustrated in FIG. 8. Depending on the psychological condition, for example stress levels,a person may experience sweating, changes in breathing rate and heart rate, changes in muscle tone, etc. Therefore, changes in physiological parameters of the human body can be observed. These changes are mainly correlated with various hernodynami changes, (e.g. changes in the amount/volume/presence of blood in various human organs, vessels and muscles). Hemodynamic changes (globally for the entire human being or locally for each body part) can be measured by observing the changes in total effective radar cross-section (EROS) of the observed person and EROS of each body part separately. The signals pertaining to these psychological parameters are compared with critical and baseline thresholds determined experimentally. Relative changes in values of all relevant observable physiological parameters can be taken into account and compared using predetermined templates or rules. A comparison is made between the observed values with a library of values defining typical various psychological conditions. The differences between the observed values and the values from the library can be used in determining the psychological condition of the observed person and in making subsequent conclusions about the possible hostile intent of the observed person.

Devices for medical use shall meet regulatory requirements as to subject exposure. An important goal for devices that require subject to be exposed to energy radiated thereby is to decrease the radiated power to admissible levels.

To decrease the power radiated by the device, a controlled key 1101 is proposed to be included in the device's circuitry that will be switching power to the transmitter. The signal will then be radiated in pulses in the frequency rise stage with a periodicity every second cycle or more frequency modulation cycles,

Another modification of the device's circuitry aimed at decreasing its radiated power is to install a digital to analog converter 1201 between the computing module and the transmitter. This will enable programmable control of the radiated power in the range from minimum maximum by issuing relevant commands to the digital to analog converter.

Yet another modification of the device's circuitry aimed at decreasing its radiated power is to install a digital to analog converter 1301 between the computing module and the control circuit of frequency deviation. This will enable programmable control of the radiated signal modulation frequency by issuing relevant commands to the digital to analog converter.

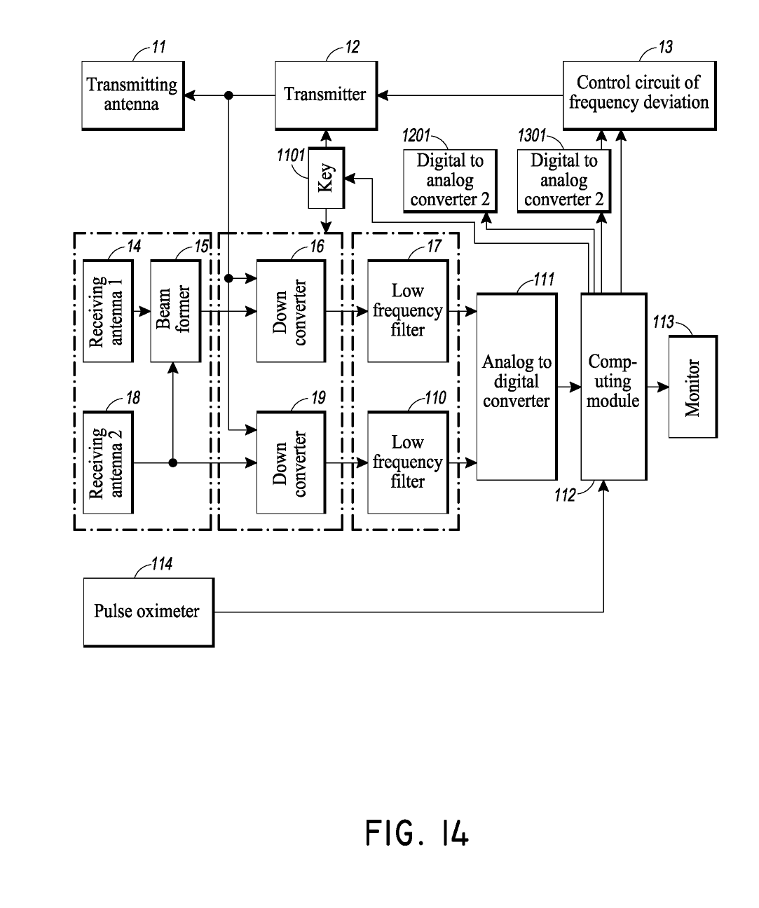

The proposed modifications of the device's circuitry can be used in aggregate, thus making it possible to significantly decrease the radiated power without affecting the device's performance. FIG. 14 illustrates the block diagram of the device, in which a controlled key 1101 provides power switching of the transmitter, a digital to analog converter 1201 between the computing module and the transmitter, and a digital to analog converter 1301 between the computing module and the control circuit of frequency deviation are installed all together.

The above description is based on the FM radar technology. However, another radar technology such as Ultra Wideband (UWB) can be used.

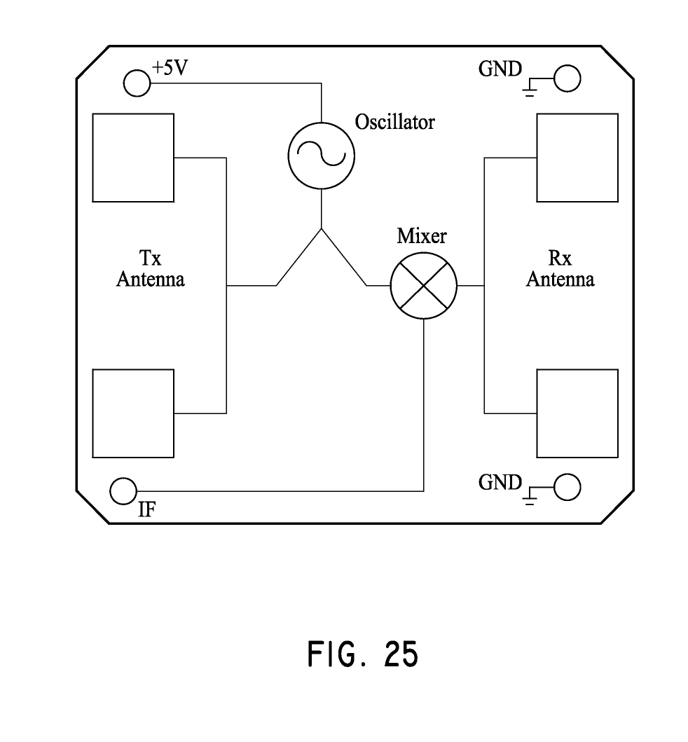

For a Doppler radar system shown in FIG. 25, a known frequency signals transmitted from an antenna which is pointed at a reference object. A separate antenna is used to receive the signal that is reflected back from the reference to measure the Doppler shift of the signal.

A simple Doppler module, also called a microwave motion sensor, can be easily integrated into the system of invention. Doppler modules have an internal oscillator used to produce the signal frequency transmitted as the source. The received signal is then mixed with this set signal, which produces an output that is a sinusoid containing the frequency difference between the output and receiver signals.

Ultra Wideband (UWB) radar systems transmit signals across a much wider frequency than conventional radar systems. The transmitted signal is significant for its very light power spectrum, which Is lower than the allowed unintentional radiated emissions for electronics. The spectrum of a very narrow-width pulse has a very large frequency spectrum approaching that of white noise as the pulse becomes narrower and narrower. These very short pulses need a wider receiver bandwith than in conventional radar systems.

The bandwidth of the UWB signal is at least 25% of the center frequency, Thus, a LIWB signal centered at 2 GHz would have a minimum bandwidth of 500 MHz and the minimum bandwidth of a UWB signal centered at 4 GHz would be 1 GHz. Often the absolute bandwidth is bigger than 1 GHz,

In most systems, these values need to be recorded or read in a tangible way and this is usually done with some sort of microcontroller. The easiest way for a microcontroller to read data from an analog device is if it outputs a DC level voltage. Some modules have this feature built into them. For those that don't, like the HB100 used in the ECE 480 Design Team 5 project, output just the AC signal. For these modules a frequency-to-voltage circuit must be implemented. An IC, such as the LM2907N, can be used for this specific purpose or any other discrete component circuit. This circuit can be used to calibrate the output data for a specific set of expected frequencies coming from the module to contain it in the reference voltage range.

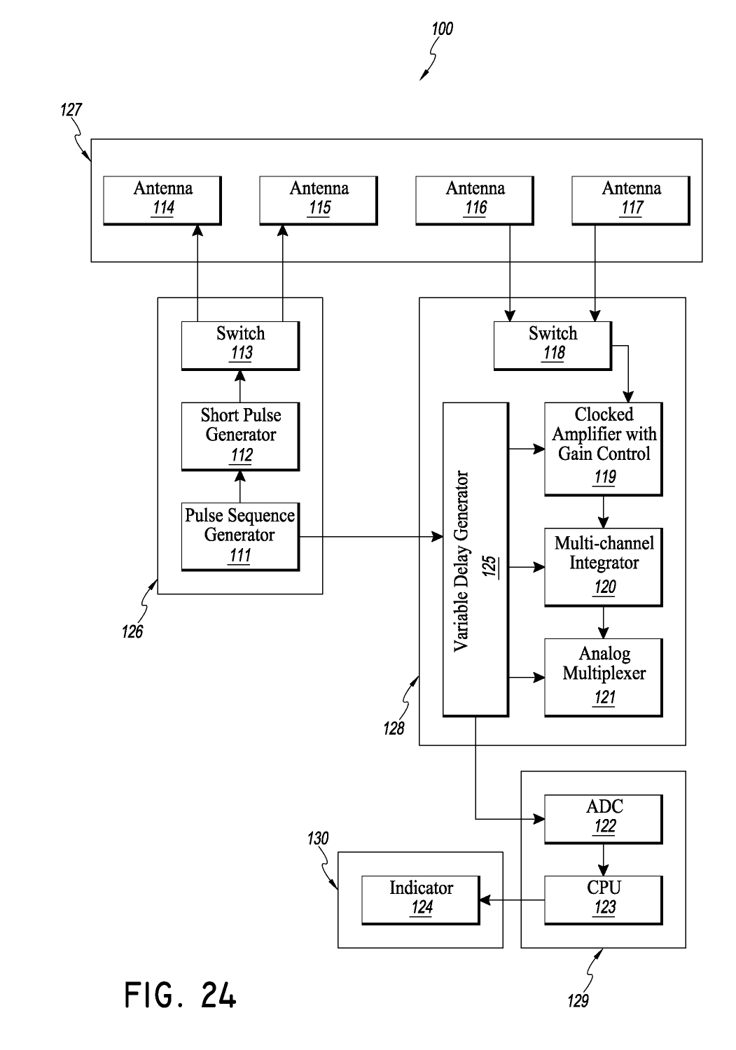

Further, an exemplary apparatus for remote non-contact determination of physiological and psychological state of individuals is described, the apparatus being based on the UWB-radar technology. As shown in FIG. 24, the apparatus 100 has signal generation unit 126, antenna unit 127, signal processing unit 128, device control unit 129, display unit 130, and power supply unit (not shown). Signal generation unit 126 includes pulse sequence generator 111, short pulse generator 112, and switch 113. Antenna unit 127 includes vertically polarized transmitting antenna 114, horizontally polarized transmitting antenna 115, vertically polarized receiving antennae 116 and 117. Signal processing unit 128 includes switch 118, clocked amplifier with gain control 119, multi-channel integrator 120, analog multiplexer 121, and variable delay generator 125. Device control unit 129 includes an analog-to-digital converter (ADC) 122 and a central processor (CPU) 123. Display unit 130 includes display 124, or other similar data display unit.

The pulse sequence generator 111 activates the short pulse generator 112 and sends the generated signal data to the variable delay generator 125 of signal processing unit 128 for further signal processing. The switch 113 selects the transmission channel to transmitting antennae 114 and 115. In one embodiment, antennae 114 and 115 transmit a train of short pulses with a specific time delay generating an ultra wideband (UWB) signal. The transmitted signal is reflected by an object, and the return signal is received by reception antennae 116 and 117. The return signal is fed to the clocked amplifier 119 having a gain control via the switch 118. The return signal is further processed by multi-channel integrator 120 and the analog multiplexer 121.

The clocked amplifier 119 boosts the signal received from switch 118. The amplifier input channel is activated only at a moment in time, allowing to receive return signals reflected from an object located at a certain distance range away. The input channel opening signal is received from the variable delay generator 125. The multi-channel integrator 120 sums the signals received by each antenna over a specified time interval to achieve a better signal-to-noise ratio. The signals received by different receiving antennae are separated using the information from the variable delay generator 125. The analog multiplexer 121 provides the multi-channel data to the ADC 122.

The clocked amplifier 119, the multi-channel integrator 120, and the analog multiplexer 121 receive the signal profile of the transmitted signal from the variable delay generator 125. Using the ADC 122, the return signal is converted to a digital signal, and the converted signal is delivered to CPU 123 of the device control unit 129, where the return signal is processed. The processed data is directed to the indicator 124 of display unit 130.

According to one embodiment, antenna unit 127 may have only one antenna for both transmitting and receiving signals, or antenna unit 127 may have one transmitting antenna 115 and one receiving antenna 116. With two receiving antennae 116 and 117, the azimuth of a target may be determined by a triangulation method. With vertically polarized transmitting antenna 114, horizontally polarized transmitting antenna 115, and two vertically polarized receiving antennae 116 and 117, richer information about the surface property and the condition of a target may be obtained to identify the target more accurately.

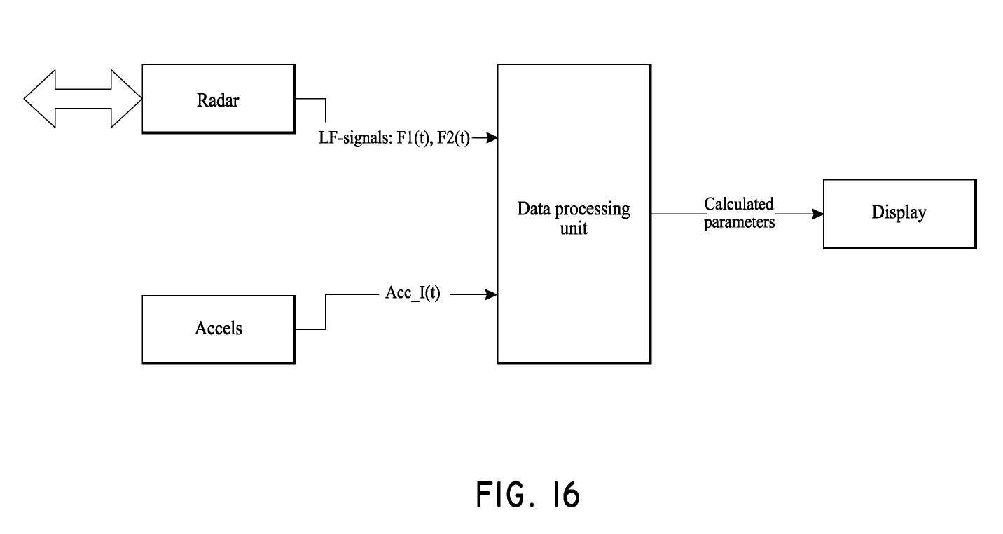

A specific embodiment of a device for determination of personal state generally referred here as vital assigns (VS) module, wherein one or more patients have accelerometers attached to their bodies is described below. In FIG. 16 a general block diagram of the VS module is presented.

In the VS module the radar emits a probing signal that rebounds from the studied objects (objects and patients in the room) and serves as input for two receiving antennae: the main antenna and the differential antenna. After processing the input signal from each of the two antennae (by mixing said signal with the emitted signal and subsequent filtering), the radar sends two following corresponding low-frequency (LF) signals serving as input for the data processing unit: main channel LF-signal (F1(t)) and differential channel LF-signal (F2(t)).

The accelerometer unit (Accels) comprises accelerometers mounted on (attached to) the patients' bodies. Signals emitted by said accelerometers (Acc_1(t)) are input into the data processing unit via a wireless communication channel. When transmitting the corresponding signal, each accelerometer transmits the ID thereof assigned to a specific patient.

The data processing unit receives LF-signals from the radar and signals from Accels, and subsequently determines the following main parameters for each patient based on said signals: a) breathing and pulse rate; b) patient's cardiac performance (by building and analyzing a plethysmogram); c) patient identification by automatically determining a match between determined parameters and each patient's ID. The obtained parameters are delivered to a display and stored in a database for each patient.

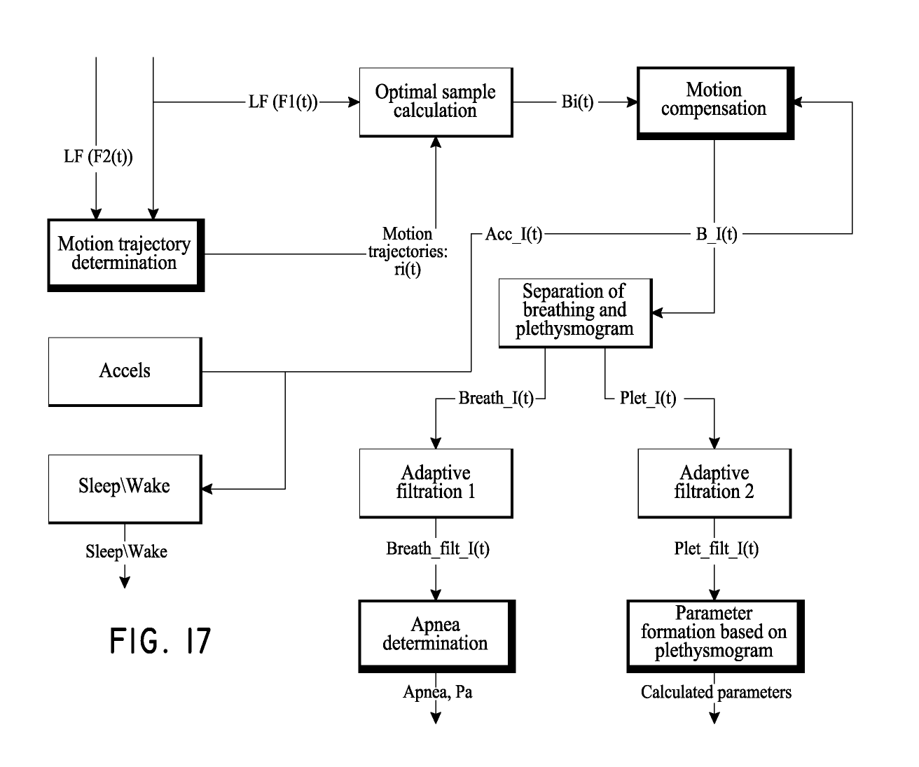

The structure of the data processing unit is shown in FIG. 17. The data processing unit is operated as follows: The motion trajectory determination unit provides the following functions: determining the presence of “breathing/moving” objects (patients) and index numbers thereof (ni) (an index number is assigned to each patient); determining current coordinates for each determined patient (Ri): and determining motion trajectory for each patient Ri(t). It must be noted that a patient's index number is a dummy parameter not yet associated with each patient's ID number in any way.

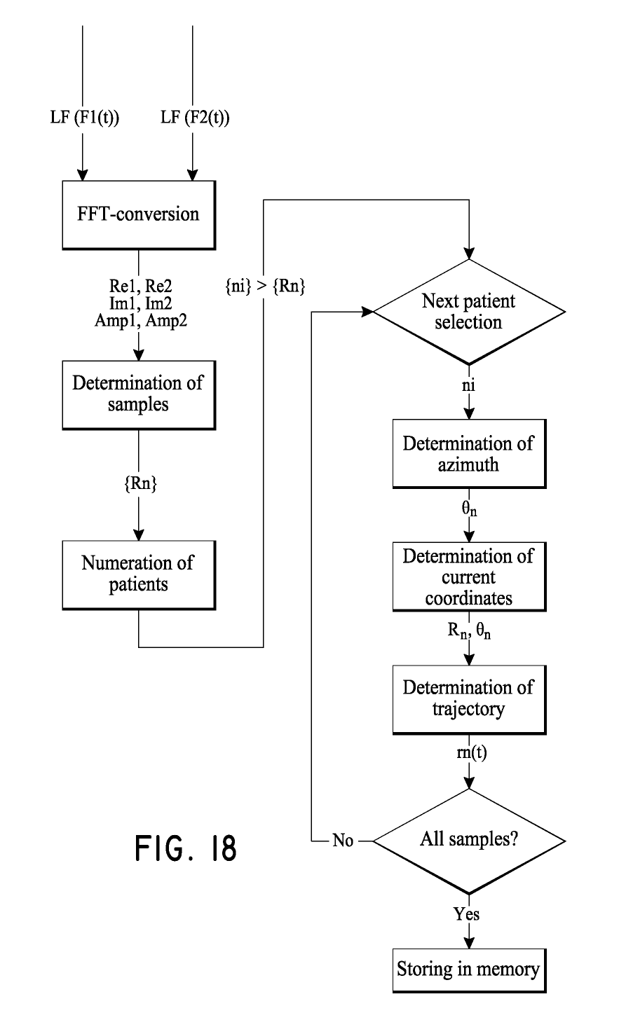

A more detailed structure of the motion trajectory determination unit is shown in FIG. 18. The optimal sample determination unit provides the following functions: a) receiving the LF-signal F1 (t) and FFT conversion thereof. The following set of parameters is determined for each range sample (Rj): Im (imaginary component), Re (real component), Apm (signal amplitude); b) the optimal sample sequence Ri,j(t) for each patient is determined based on the motion trajectory of each patient ri(t) received from the motion trajectory determination unit. Said sequence contains index numbers of time-variant samples during which the signals received from a patient possess the best characteristics in terms of resolving power and determination of breathing and cardiac activity); c) selection of an optimal signal from the available set (Re, Im, Amp) for the selected sample is performed for each patient.

A signal with maximum standard deviation value over the set monitoring period T is selected from the obtained values (Im, Re, Amp). In other words, the following values are determined; {STD(Im), STD(Re), STD(Amip)}

The value of the resulting signal corresponds to Im, Re, Amp (taking into account the STD maximum value). The operational result of the unit is the signal (Bi(t)), where i is the patient's index number. The signal contains the “totalmotion” (motion, breathing and cardiac activity) for each patient,

The motion compensation unit provides the following functions: a) determining parameters describing motion of a patient based on the optimum signal Bi(t) (for each patient with an index number); b) determining motion for each patient with an identification number (ID) based on signals received from accelerometers; c) establishing a match between the patient's index number and his ID; d) establishing motion parameters for identified patients; e) compensating for voluntary movements in the general signal Bi(t) and forming signal (b_l(t)) containing only “useful” motion (breathing and cardiac activity). Said compensation is performed by comparing data Bi(t) received from (and processed by) data Acc_l(t) received from accelerometers.

The operational result of the unit is a list of patient IDs (nl) with a signal (b_l(t)) compensated for voluntary movements matched to each patient. Said signal generally contains “relevant” movements (breathing and cardiac activity), whereas signals associated with so-called “voluntary” movements (due to the patient walking or moving parts of the body (arms, legs, head, etc) are removed from said signal.

The Accels unit forms and transmits to the motion compensation unit the following data for each patient provided with said accelerometers: a) the patient's identification number (nl). Said number can be associated with patient's name or any other information that allows the physician to unambiguously identify said patient; and b) motion signals for said patient caused by his or her motion or movement of his or her body parts (arms, legs, head, etc.): Acc_l(t).

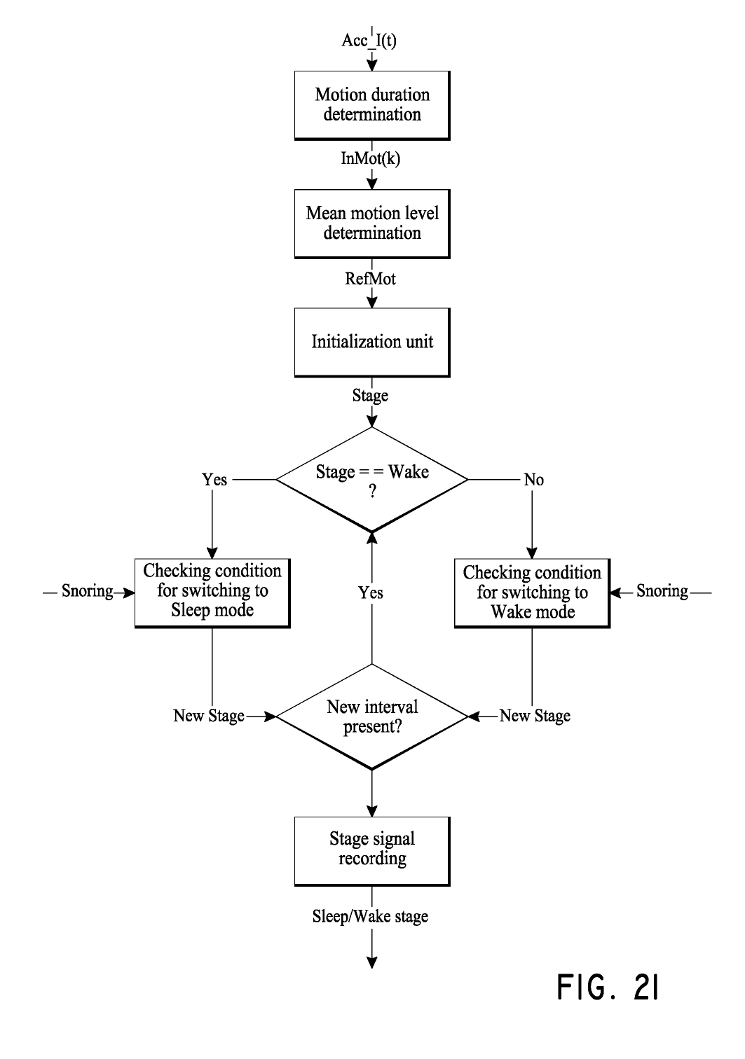

The Sleep/Wake unit determines whether the patient is asleep (Sleep) or awake (Wake). Said determination is performed based on data received from the accelerometer unit Acceli(t). The more detailed structure of said unit is shown in FIG. 21.

The breathing and plethy nog a n separation unit separates the (b_l(t)) signal received from the motion compensation unit into two categories: a) signal used to determine breathing (Breath_l(t)); and b) signal used to determine parameters of cardiac activity (Plet_l(t)) and to build various plethysmograms based thereon.

Signal separation is performed using digital filtering methods: the input signal (b_(t)) is passed through barrier filters corresponding to breathing or cardiac activity.

The adaptive filtering unit 1 performs filtering of the input signal Breath_l(t) by readjusting filter parameters taking into account current frequency characteristics of the input signal:

a) first, the input signal is filtered for a “general case”, wherein frequency characteristic can fluctuate within a broad range. Based on this step, the adjustment of the frequency domain of the actual input signal is performed;

b) filtering within a narrower domain taking into account the previously determined signal fluctuation frequency range.

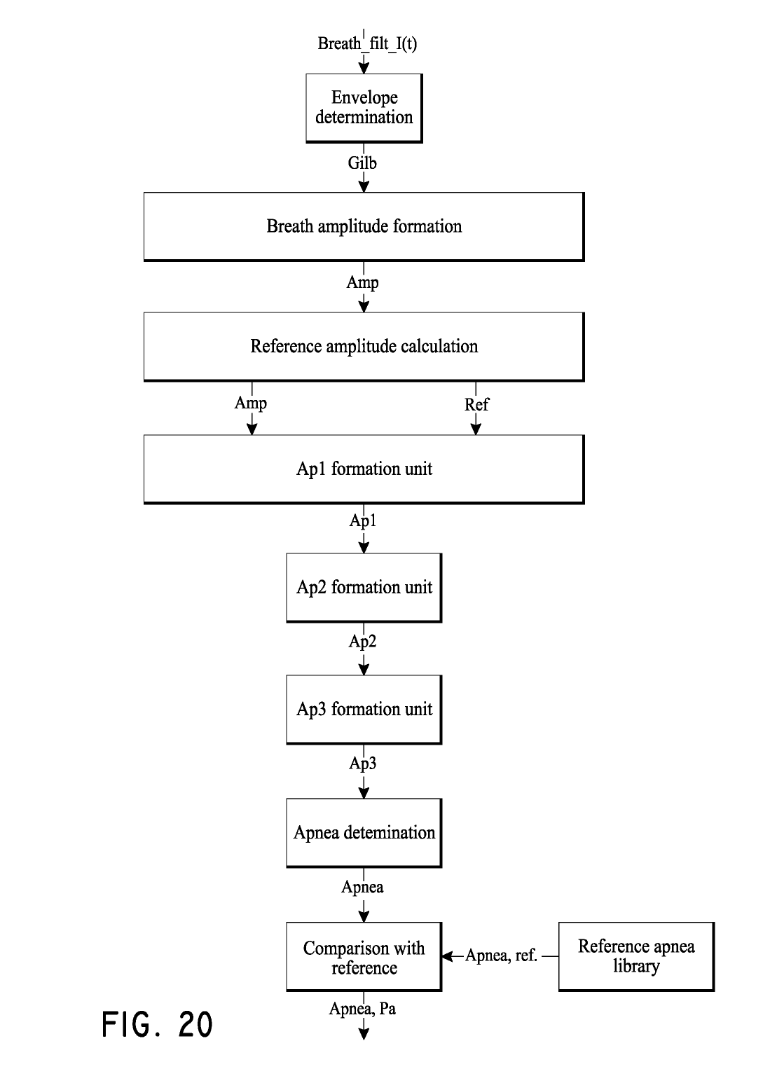

The apnea determination unit determines apnea occurrences for each patient from the received input signal Breath_l(t). The more detailed structure of said unit is shown below in FIG. 20.

The adaptive filtering unit 2 performs filtering of the input signal Plet I(t) by readjusting filter parameters taking into account current frequency characteristics of the input signal:

a) first, the input signal is filtered for a “general case”, wherein frequency characteristic can fluctuate within a broad range. Based on said step, the adjustment of the frequency domain of the actual input signal is performed;

b) filtering within a narrower domain taking into account the previously determined signal fluctuation frequency range.

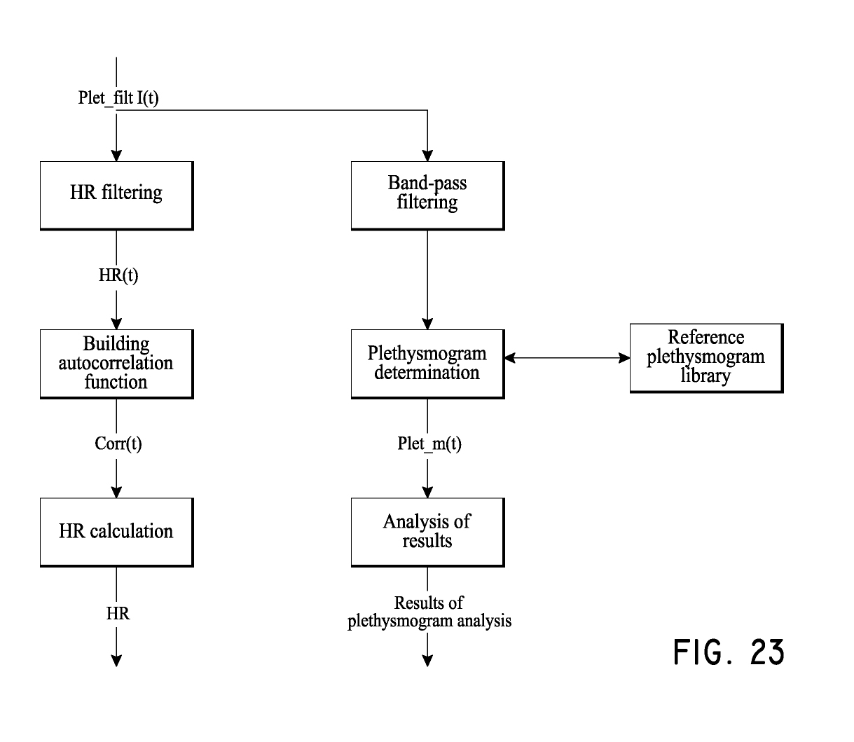

The plethysprogram-based parameter formation unit determines the following parameters for each patient. The detailed structure of this unit is presented in FIG. 23.

FIG. 18 shows the detailed structure of the motion trajectory determination unit. The motion trajectory determination unit is operated as follows: The FFT conversion unit receives low frequency signals (F1(t) and F2(t)) and performs FFT conversion thereof. The result of said conversion is a set of the following signals for each type (F1 or F2) and for the range sample Rj:

Re1, Re1—actual parts;

Im1, Im2—imaginary parts;

Amp1, Amp2—amplitudes.

The moving sample determination unit analyzes each determined sample (Rj) and determines samples in which motion is observed. For that purpose,standard deviation of the signal for each of the two channels is determined for each sample over the monitoring period ΔT : a) main channel: STD(Re1), STD(Im1), STD(Amp1), and b) differential channel; STD(Re2), STD(Im2), STD(Amp2).

Then a maximum deviation degree is determined for each channel: a) main channel: max_{—}1=max(STD(Re1), STD(Iml), STD(Amp1)); and b) differential channel: max_{—}2=max(STD(Re2), STD(Im2), STD(Amp2)). If max_{—}1 and max_{—}2 exceed the set threshold P: max_{—}1>P and max_{—}2>P, then the decision is made that the sample Rj contains the patient. This analysis is successively performed for all determined samples. The result of this process is a list of samples {Rn} presumably containing the patient.

The patient numbering unit provides successive numbering of all determined samples exceeding set movement level threshold and containing patients. The result of said process is a match between the patient's index number (ni) and the number of range sample {Rn; containing said patient.

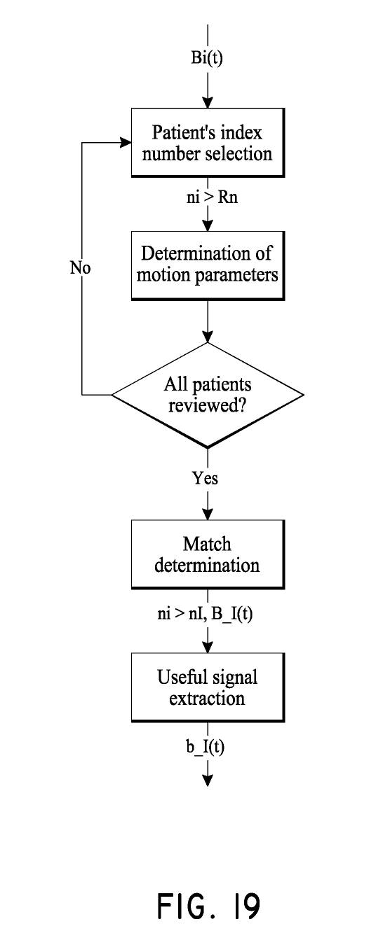

It must be noted that said numbering is the dummy numbering. In other words, a patient ni can be any patient present in the room. The system is not yet capable of establishing a match between the patient's index number and his or her identification number (specific patient's ID- nl). Said process (matching the patient's index number and hls or her ID) will be performed by the motion compensation unit; the structure thereof is shown below in FIG. 19.

The next patient selection unit selects the next index number (ni) of a patient whose current coordinates and motlon trajectory are to be determined.

The patient's azimuth determination unit perfo is the follo ing steps: a) choosing amplitudes for the main and differential channels for a set sample Rn: Ampin, Amp2n: and b) determining the patient's azimuth On in said sample with respect to the radar, based on the following formula:

$$θ = arc sin ( \frac{arc sin ( - \frac{H 2}{H 1} )}{π d} ) ,$$

where H2=Amp2n, H1=Amp1n, and d is the distance between antennae of the main and differential channels.

The current patient coordinates deter i ation unit determines coordinates as a set consisting of the azimuth value On and the current range sample value Rn, vithich determines the distance between the patient and the radar.

The operational result of this process is the determination of polar coordinates (Rn, θn) of a patient with index number ni.

The patient trajectory determination unit determines the trajectory of patient's movement over the monitoring period AT. For this purpose, all previously determined and collected coordinates for the patient with index number ni are collected. The operational result of said process is the determination of patient's movement trajectory over the monitoring period ΔT.

The sample enumeration evaluation unit determines whether all determined samples had been analyzed. If not all of the samples had been analyzed (i.e. samples containing movement are still present), the process is switched to the patient numbering unit to determine parameters for the patient with the next index number,

If all patients' index numbers have been reviewed, the process is switched to the memory storage to store determined trajectories for each patient. The memory storage unit stores current coordinates arid calculated motion trajectories for each patient with an index number.

FIG. 19 shows a schematic diagram of the motion compensation unit. The motion compensation unit is operated as follows:

The patient index number selection unit selects the next patient's index number {ni}, the corresponding range sample {Rn} and signals (Im, Re, Amp).

The motion parameters determination unit determines motion characteristics for the patient with a given index number. The above is achieved by:

determining time intervals with motion level exceeding a set threshold from signals Im, Re, Amp;

determining a frequency spectrum for the determined time intervals based on FFT; said spectrum representing a characteristic of patient's movement over said time interval;

storing the obtained movement characteristics (for said patient and the determined time interval) in a data library.

The complete patient enumeration condition check unit determines the unit transition conditions. If patients with undetermined motion parameters remain, the process is switched to the patient index number selection unit. Otherwise, the process is moved on to the next unit.

The match determination unit establishes a match between the patients' index numbers and their identification numbers (ID). For this purpose, the following sequence f actions is performed:

All combinations of previously determined patients' index numbers (nj) and ID numbers (nl) set using accelerometers are checked. For example, the index number ni is matched with the identification number ID1, n2 is matched with ID2, etc., up until matching nN with IDN. Following is a simple example with three patients. In this case, the following combinations are present:
combination_{—}1. n1-ID1; n2-ID2; n3-ID3;
combination_{—}2: n1-ID1; n2-ID3; n3-ID2;
combination_{—}3: n1-ID2; n2-ID1; n3-ID3;
combination _{—}4: ID2; n2-ID3; n3-ID1;
combination _{—}5: n1-ID3; n2-ID1; n3-D 2;
combination_{—}6: n1-ID3: n2-ID2; n3-ID1.

Generally, N! combinations exist for N patients. For each combination, a degree of match between the patients' index numbers and their ID numbers is determined as follows:
time intervals for the corresponding sample are selected for each pair(nj, IDi), said time intervals previously determined by the motion parameters determination unit. For the determined time interval, a frequency spectrum is determined using FFT; said spectrum is a characteristic of motion determined based on radar readings, said characteristic corresponding to the patient with the index number nj;
accelerometer readings are selected for the said interval and the corresponding IDi. Said readings (for the determined time interval) are integrated twice (to change from acceleration parameters to patient removement parameters). The obtained frequency spectrum is a characteristic of motion determined based on accelerometer readings, said characteristic corresponding to the patient with identification number IDi;
the degree of match values are calculated as a degree of correlation between frequency spectrums obtained based on radar and accelerometer readings.
from the evaluations obtained during previous step, a combination with a maximum degree of match value is selected. This combination determines the match between the patient's index number and his ID; ni->ni.

The operational result of the said unit is the determination of match between the ID of a specific patient (nI) and the main parameters of said patient: current coordinates (Rn, en), motion trajectory rn(t), the value of the determined signal (B_l(t)).

The useful signal determination unit forms the useful signal (b_l(t)) predominantly comprising oscillations caused by breathing and cardiac activity. For that purpose, the patient's voluntary movements (patient's actual movement, movement of body parts, etc.) are subtracted (compensated) from the input signal (BKt)) determined from the accelerometer readings. The unit performs for each patient determination of intervals with motion level exceeding a set threshold P0;

Further, the unit performs for each patient the signal (B_(t)) adjustment in the frequency domain:

a) the second integration of readings corresponding to the current ID of accelerometers for determined intervals;

b) the determination of a “voluntary” motion signal based on FFT accelerometer readings determined for the corresponding ranges;

c) creating a spectrum for the determined range for (B_l(t));

d) adjustment of the spectrum (created at the step c) based on the “voluntary” motion spectrum for the said range obtained during step b as follows: each harmonic of the “useful signal” spectrum (B_l(t) is multiplied by the weight coefficient inverse to the “voluntary” motion signal harmonic value, said signal obtained during step b;

e) transition from the adjusted “useful signal” (B_l(t)) spectrum to the time domain.

Further, the unit performs for each patient filtering the obtained signals using a band-pass filter that adapted for frequency range of breathing and cardiac activity. The operational result of the said unit is the signal (b_l(t)) predominantly containing “useful” oscillations caused by the breathing motions and the cardiac activity,

FIG. 20 shows a schematic diagram of the apnea determination unit. The input signal envelope determination unit provides envelope determination from the input signal Breath_filt_l(t) based on the Hilbert transform:

$$H ( u ) ( t ) = - \frac{1}{π} {lim}_{∈ \to 0} {\int }_{ε}^{∞} \frac{u ( t + τ ) - u ( t - τ )}{τ} d τ .$$

The breath amplitude formation unit provides smoothing of the envelope values obtained in the previous step by interpolating envelope values between local maxima,

The reference amplitude calculation unit provides calculation of the reference breath amplitude (Ref), and the apnea determination is later carried out with respect thereto, Ref value is calculated for the current interval between successive motions. In this case, Ref=K1*mean(Amp) or Ref=K2*max(Amp), K1 and K2 values in this case are chosen based on results of preliminary tests and the subsequent comparison of obtained results with reference device readings.

The Ap1 formation unit: determination of potential apnea occurrences. Ap1, Ap2, Ap3 are signals used for determining apnea patterns (intermediate potential apnea occurrences). Subsequent corrections are then made to determine whether said signals correspond to apnea occurrences. Ap1 is the result of Ref amplitude analysis. In this case, Ap1=1 if the current breath amplitude exceeds a certain threshold: Amp>K3*Ref, Ap1=−1 if Amp <K4*Ref. In all other cases: Ap1=0.

The operational output of the unit comprises presenting Ap1 in a square waveform (+1, 0, −1). In this case, <<−1>> denotes the apnea occurrence interval.

The Ap2 formation unit. Ap2=Ap1 The unit provides rejection of overly long potential apnea intervals with the value less than <<1>> (<<0>> or <<−1>>) and the duration of over 60 seconds. The operational output of the unit allows to increase apnea determination confidence.

The formation unit. Ap3=Ap2, with the exception of motion intervals. For motion intervals, Ap3=1. The unit provides removal of motion intervals.

The Apnea determination unit. Apnea=Ap3 with the exception of intervals, in which Ap3<1 and the duration of which is less than 10 seconds (minimum apnea duration). For said intervals, Apnea=1.

The formed Apnea signal indicates the presence of the clear patterns characteristic for breathing disorders if the value is <<−1; and the presence of less clear apnea patterns (with lower confidence) if the value is 0.

The reference comparison unit compares the breathing signal (Breath) for a time interval corresponding to the determined Apnea occurrence with an array of reference apnea occurrences selected from a reference apnea library. The library is composed based on test measurements, in which the results of apnea determination based on the Breath signal are compared with readings from reference devices which determine various apnea types. The so-called “golden standard of somnology” can be used as said reference devices. By comparing the current Breath signal with reference apnea occurrences, the match rate with each of the reference occurrences is determined. The possibility (Pa) that the determined event is an apnea occurrence is determined based on the maximum match rate value of all match rate values.

FIG. 21 shows the structure of the Sleep/Wake determination unit. The unit includes:

The motion duration determination unit. The whole monitoring period is split into N-minute long intervals. Based on the analysis of the input Acc_l(t) signal for each current interval k, the time interval, during which the signal level exceeded set threshold (P2) is determined. In this case, it is considered that the patient had moved. The obtained combined motion time is designated as InMot(k).

The mean motion level determination unit determines the mean motion time over the whole monitoring period T: RefMot. Based on RefMot and the initial calibration, two parameters are determined: K1, K2 (K1<K2). In this case,

K1*RefMot is a threshold value for switching to a Sleep mode;

K2*RefMot is a threshold value for switching to a Wake mode.

The initialization unit. For the first several minutes of the recording, the Stage value is set as equal to the Wake value. Condition for switching to Sleep mode: If InMot <K1*RefMot, then Stage=Sleep.

Condition for switching to Wake mode. If nMot >K2* ReflMot, then Stage=Wake.

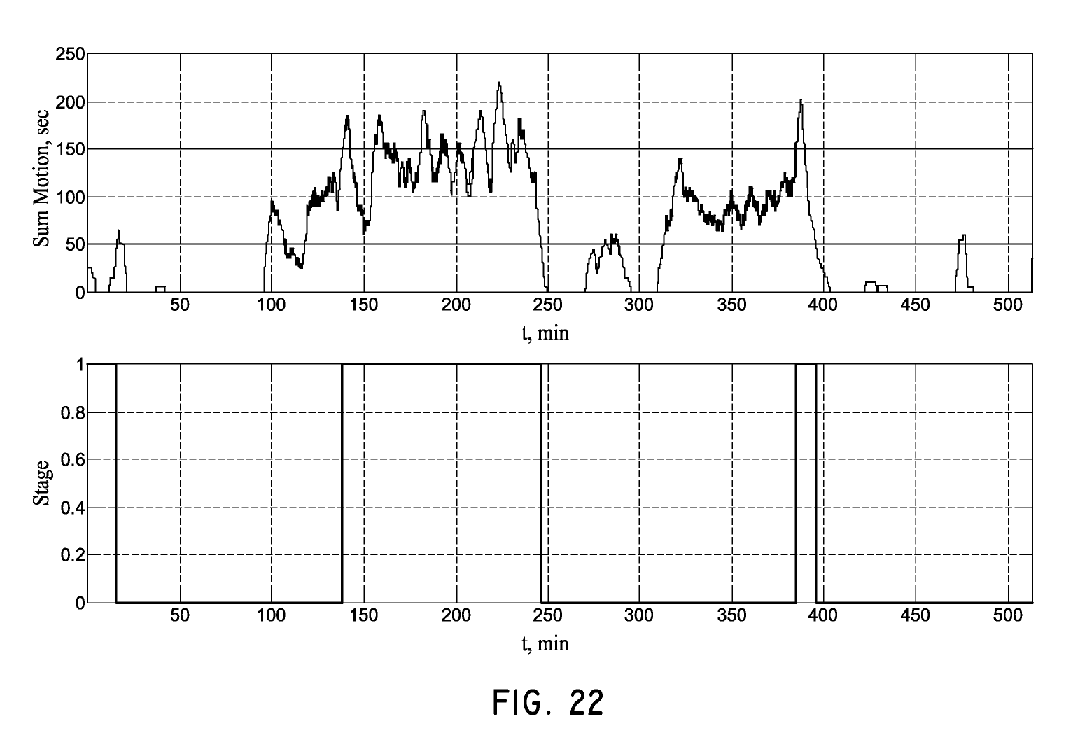

Operational results of the Sleep/wake and initialization units are shown in FIG. 22. The upper graph represents the InMot(k) signal (see FIG. 7). The lower graph represents Wake (=1) and Sleep (=0) signals obtained in accordance with processes described with reference to these units. The upper green line on the upper graph corresponds to K2*RefMot value, and the lower red line corresponds to K1*RefMot value.

Checking stage: Is the next interval present or are all intervals reviewed? The condition check is performed until all N-minute intervals of the recording have been fully reviewed.

The status storage unit. The unit stores the obtained status (Sleep/Wake) for each patient.

FIG. 8 shows the structure of the plethysmogram-based parameter formation unit. The unit operates as follows:

The heart rate filtering unit processes the input signal Plet_filt_(t) to extract the signal associated with heart rate (HR(t)).

The autocorrelation function creation unit provides the autocorrection function (Corr(t)) for the input signal HR(t) to extract periodical signals present in the said input signal therefrom.

The heart rate calculation unit extracts heart rate values from the autocorrelation function.

The band-pass filter unit performs band-pass filtering of the input signal (Plet_filt_l(t)) to extract the following rhythmic processes of the peripheral hemodynamics therefrom:

a) superpulse wave (frequency range: over 1.7 Hz);

b) pulse wave (frequency range: 0.8-1.1 Hz);

c) high-frequency breathing wave (frequency range: 0.17-0.33 Hz);

d) slow y-rhythm wave (frequency range: 0.13-0.15 Hz);

e) vasomotor p-rhythm wave (frequency range: 0.06-0.12 Hz);

f) α-rhythm (frequency range: 0.017-0.05 Hz);

g) ω-rhythm (frequency range: 0.0017-0.008 Hz).

The plethysmogram determination unit determines the plethysmogram corresponding to the set rhythmic process of the peripheral hemodynamics by comparing the current signal with the reference plethysmogram library.

The result analysis unit forms and outputs the results of piethysmogram analysis.

The following symbols are used in the above description:
ni—patient's index number.
nl—patient's identification number.
Rn θn—current coordinates of the patient
rn(t)—current trajectory of the patient
Rj—range sample
Ri,j—optimal sample sequence
Bl(t)—optimal noisy signal from the patient
bl(t)—optimal filtered signal from the patient
Accell (t)—signal from accelerometers mounted on the identified patient's body
Δt—LF-signal interval;
ΔT—STD determination interval for Re, Im, Amp,
N—number of patients
ΔRn}—samples containing patients

What is claimed is:

1. An apparatus for remote monitoring physiological parameters, comprising: a radar transmitter having a transmitting antenna for radiation of a radio frequency signal towards at least one human body, and at least one radar receiver for receiving a signal reflected from the at least one human body, the radar receiver comprising a receiving antenna positioned at a predefined distance from the transmitting antenna, the apparatus further comprising at least one accelerometer adapted to be placed on a human body, and a signal processor, wherein respective outputs of the radar receiver and accelerometer are connected to the input of the signal processor which is configured for extracting and processing physiological parameters of the at least one human body from the inputted signals of the receiver and accelerometer.

2. The apparatus of claim 1, wherein the accelerometer contains a wireless transmitter,the signal processor contains a wireless receiver, and the output of the accelerometer is connected to the input of the signal processor through a wireless communication channel.

3. The apparatus of claim 1, further comprising at least two radar receivers, wherein their respective receiving antennae are positioned at a predetermined distance from one another and from the transmitting antenna.

4. The apparatus of claim 1, wherein the radar receiver comprises a clocked amplifier having its input connected to the receiving antenna.

5. The apparatus of claim 1, wherein the transmitter is adapted to produce frequency modulated signals in the form of a train of pulses with a predefined delay between pulses.

6. The apparatus of claim 5, wherein the pulses have duration of half a period of modulation frequency variation.

7. The apparatus of claim 1, wherein the signal processor has a control output, which is connected to the digital input of a digital-to-analog converter, the radar transmitter has a frequency deviation control input, and the analog output of the digital-to-analog converter is connected to the frequency deviation control input of the radar transmitter.

8. A method for determination of the distance from each receiving antenna to a body using the apparatus of claim 1, wherein the emitted frequency and the received frequency are measured at the same moment, the difference between these frequencies is multiplied to the modulation frequency sweep period, and divided by the modulation frequency swing, and the result is scaled by the multiplication to one fourth of the speed of light in the air.

9. A method for determination of the azimuth to each body of the apparatus of claim 1, wherein a phase shift is measured between the harmonic components selected for the same human body, received from two receiver channels; and the required azimuth is obtained as the arc sine of the said phase shift multiplied to the radar wavelength and divided by the distance between the receiving antennae.
